(19) United States
(12) Patent Application Publication
(10) Pub. No.: US 2018/0214341 A1
(43) Pub. Date: Aug. 2, 2018

(54) Passive Anaerobic and Aerobic Exercise Assist System for the Inactive and Sedentary Population

(71) Applicant: NAINAR AMALAN PRADEEP JEYAPRAKASH, Chennai (IN)

(72) Inventor: NAINAR AMALAN PRADEEP JEYAPRAKASH, Chennai (IN)

(21) Appl. No.: 15/684,901

(22) Filed: Aug. 23, 2017

Related U.S. Application Data
(60) Provisional application No. 62/378,817, filed on Aug. 24, 2016.

(51) Int. Cl. A61H 36/00; A61H 1/00; A61H 9/00; A61F 7/00; A61N 1/04; A61N 1/36

(52) U.S. Cl. CPC A61H 36/00; A61H 1/00; A61H 9/0092; A61F 7/0097; A61N 1/0456; A61N 1/0452; A61H 2201/1238; A61F 7/007; A61H 2201/0207; A61H 2201/10; A61H 2203/0456; A61H 2201/1645; A61H 2201/5007; A61N 1/36014

(57) ABSTRACT

A passive anaerobic and aerobic exercise assist system (100) is provided. The system (100) comprises an External Counter Pulsation (ECP) unit (110), a perspiration unit (120) connected with ECP unit (110), an Electronic Muscle and Nerve Stimulation (EMS/TENS) unit (130) connected with the perspiration unit (120), a monitoring device (140) connected with the ECP unit (110), the perspiration unit (120) and the EMS/TENS unit (130).

### CROSS REFERENCE TO RELATED APPLICATION

The present application claims priority to U.S. patent application No. 62/378,817 filed on Aug. 24, 2016.

### TECHNICAL FIELD

The present disclosure, generally, relates to exercises and physical health care and more particularly relates to a passive anaerobic and aerobic exercise assist system for sedentary and physically less active population.

### BACKGROUND

A sedentary lifestyle is a type of lifestyle with no or irregular physical activity. A person who lives a sedentary lifestyle may colloquially be known as a couch potato. It is commonly found in both the developed and developing world. Sedentary activities include sitting, reading, socializing, watching television, playing video games, and computer use for much of the day with little or no vigorous physical exercise. A sedentary lifestyle can contribute to many preventable causes of death. Screen time is the amount of time a person spends watching a screen such as a television, computer monitor, or mobile device. Excessive screen time is linked to negative health consequences. A sedentary lifestyle and lack of physical activity can contribute to or be a risk factor for anxiety, cardiovascular disease, colon cancer, depression, diabetes, high blood pressure, lipid disorders, mortality in adults, obesity, osteoporosis, scoliosis, spinal disc herniation (lower back pain) etc.

USA In the 2008 United States American National Health Interview Survey (NHIS) 20 36% of adults were considered inactive. 59% of adult respondents never participated in vigorous physical activity lasting more than 10 minutes per week. Percentage of adults who self-report doing no physical activity or exercise other than their regular job in the last 30 days—The prevalence of physical inactivity ranges from a high of 31.6% of adults in Mississippi to 16.4% of adults in Colorado. The 25 national median of adults who do not engage in physical activity outside of their regular job is 22.6%, a slight decrease from 23.5% of adults in the 2014 Edition. United States Physical Inactivity (1997-2015)—The 2015 ranks are based on self-report data from CDC 2014 Behavioural Risk Factor Surveillance System (BRFSS). In the 2012 Edition, physical inactivity was referred to as sedentary lifestyle.

Globally, around 31% of adults aged 15 and over were insufficiently active in 2008 (men 28% and women 34%). Approximately 3.2 million deaths each year are attributable to insufficient physical activity. In 2008, prevalence of insufficient physical activity was highest in the WHO Region of the Americas and the Eastern Mediterranean Region. In both these regions, almost 50% of women were insufficiently active, while the prevalence for men was 40% in the Americas and 36% in Eastern Mediterranean. The South East Asian Region showed the lowest percentages (15% for men and 19% for women). In all WHO Regions, men were more active than women, with the biggest difference in prevalence between the two sexes in Eastern Mediterranean. This was also the case in nearly every country.

According to the study to assess physical activity patterns across India—as part of the Indian Council of Medical Research-India Diabetes (ICMR-INDIAB) study, that overall 392 million individuals are inactive in India. This is a staggering figure and implies a huge population at risk for developing diabetes and other non-communicable diseases. This underscores the urgent need to improve overall physical activity levels with specific reference to recreational physical activity. The study shows that a large percentage of people in India are inactive with fewer than 10% of the Indian population engaging in recreational physical activity. Therefore, urgent steps need to be initiated to promote physical activity to stem the twin epidemics of diabetes and obesity in India.

As a response to concerns over health and environmental issues, some organizations have promoted workplace interventions such as alternative activity workstations, sit-stand desks, promotion of stair use are among measures being implemented to counter the harms of sedentary workplace environments. A Cochrane systematic review published in 2016 concluded that “at present there is very low quality evidence that sit-stand desks can reduce sitting at work at the short term. There is no evidence for other types of interventions.” Also, evidence was lacking on the long term health benefits of such interventions. Despite the well-known benefits of physical activity, many adults and many children lead a relatively sedentary lifestyle and are not active enough to achieve these health benefits.

Therefore, there is a need in the art for a passive anaerobic and aerobic exercise assist system which ameliorates all or some of the deficiencies of the prior art and help users in taking care of their bodies, practically without any or too little anaerobic or aerobic exercise activities, by simulating comprehensive and all round “Passive Exercise”.

### SUMMARY

According to a first aspect of the present disclosure, there is provided a passive anaerobic and aerobic exercise assist system. The system comprises an External Counter Pulsation (ECP) unit, the ECP unit includes a first computing device, a first control module connected with the first computing device, an air supply module connected with the first control module, a first power module connected with the air supply module and the first computing device and an implementation section connected with the air supply module, the implementation section having a plurality of cuffs and a plurality of connection tubes connecting the respective plurality of cuffs to the air supply module, a perspiration unit connected with ECP unit, the perspiration unit includes an heat output zone, the heat output zone further having a first housing, for receiving a head of the user and a second housing connected with the first housing, for receiving a body of the user, a thermal blanket provided in the heat output zone, a heating element disposed in the thermal blanket and a power control box connected with the heating element, an Electronic Muscle and Nerve Stimulation (EMS/TENS) unit connected with the perspiration unit, the EMS unit includes a third power module, a third control module connected with the third power module and an output zone connected with the third control module and a monitoring device connected with the ECP unit, the perspiration unit and the EMS/TENS unit. Further, the first power module is configured to supply electrical power to the air supply unit and the first computing device. And the first computing device is configured to operate the air supply module. Also, the air supply unit is configured to supply pressurised air to the plurality of cuffs via the respective plurality of connection tubes when instructed by the first computing device and extract pressurised air from the plurality of cuffs via the respective plurality of connection tubes when instructed by the first computing device. Moreover, the first control module is configured to regulate a temperature, an air pressure and a quantity of air supplied from the air supply module. And the plurality of cuffs are configured to wrap around a body part of the user, inflate on receiving the pressurised air and apply pressure on the body part of the user and deflate after the pressurised air is extracted from the plurality of cuffs and release the pressure on the body part of the user. Further, the power control box is configured to supply electrical power to the heating element and accordingly control the temperature of the heating element and the amount of heat radiated from the heating element. Also, the heating element is configured to heat up and provide heat in the heat output zone to the user. The thermal blanket is configured to prevent direct contact of the user to the heating element and the third power module is configured to provide electrical current supply to the third control module. Moreover the third control module is configured to modulate a pulse width, intensity and frequency of the current supplied and transmit the modulated pulse to the output zone and the output zone is configured to transmit the modulated pulse to the user for nerve and muscle stimulation.

In accordance with an embodiment of the present invention, the first control module comprises a plurality of sensors connected to the first computing device, a plurality of valves connected to a pressure tank and the first computing device and a pressure regulator connected to the plurality of valves.

In accordance with an embodiment of the present invention, the air supply module comprises an air compressor and a muffler connected to the compressor.

In accordance with an embodiment of the present invention, the first power module comprises a power supply, a power panel connected to the power supply, a transformer connected to the power panel and a cooling fan.

In accordance with an embodiment of the present invention, the thermal blanket comprises of a first layer of heating element, a second layer of organic carbon fibre adjacent to the first layer of heating element and a layer of nylon covered by a layer of Polyurethane (PU) fabric wrapped around the first layer and the second layer, thereby forming a blanket.

In accordance with an embodiment of the present invention, the heating element comprises a plurality of copper electrodes.

In accordance with an embodiment of the present invention, the third control module comprises a Digital to Analog Convertor (DAC), a stimulation current control device connected with the DAC, a micro-controller connected with a stimulation current control device, a communication module connected with the micro-controller and a stimulation pulse width and frequency generator connected with the stimulation current control device.

In accordance with an embodiment of the present invention, the output zone comprises a plurality of output channels.

### BRIEF DESCRIPTION OF THE DRAWINGS

So that the manner in which the above recited features of the present disclosure can be understood in detail, a more particular description of the disclosure, briefly summarized above, may have been referred by embodiments, some of which are illustrated in the appended drawings. It is to be noted, however, that the appended drawings illustrate only typical embodiments of this disclosure and are therefore not to be considered limiting of its scope, for the disclosure may admit to other equally effective embodiments.

These and other features, benefits, and advantages of the present disclosure will become apparent by reference to the following text figure, with like reference numbers referring to like structures across the views, wherein:

FIG. 1 illustrates a passive anaerobic and aerobic exercise assist system, in accordance with an embodiment of the present invention;

FIG. 2A illustrates an External Counter Pulsation (ECP) unit of the system, in accordance with another embodiment of the present invention;

FIG. 2B-2C illustrate an implementation section of the ECP unit, in accordance with an embodiment of the present invention;

FIG. 3A illustrates a perspiration unit and an Electronic Muscle and Nerve Stimulation (EMS/TENS) unit of the system, in accordance with another embodiment of the present invention;

FIG. 3B illustrates an output zone of the perspiration unit, in accordance with embodiment of the present invention; and

FIG. 3C illustrates a thermal blanket of the perspiration unit, in accordance with embodiment of the present invention.

FIG. 3D-3E illustrate electrode placement charts of improved muscle and nerve stimulation (EMS/TENS) unit.

### DETAILED DESCRIPTION OF THE EMBODIMENTS

Detailed embodiments of the present disclosure are described herein; however, it is to be understood that disclosed embodiments are merely exemplary of the present disclosure, which may be embodied in various alternative forms. Specific process details disclosed herein are not to be interpreted as limiting, but merely as a representative basis for teaching one skilled in the art to variously employ the present disclosure in any appropriate process.

The terms used herein are for the purpose of describing exemplary embodiments only and are not intended to be limiting. As used herein, the singular forms “a,” “an,” and “the” are intended to include the plural forms as well, unless the context clearly indicates otherwise. It will be further understood that the terms “comprises” or “comprising,” when used in this specification, do not preclude the presence or addition of one or more components, steps, operations, and/or elements other than a mentioned component, step, operation, and/or element.

The embodiments of the present disclosure will now be described more fully hereinafter with reference to the accompanying drawings, which form a part hereof, and which show, by way of illustration, specific example embodiments. The following detailed description is not intended to be taken in a limiting sense.

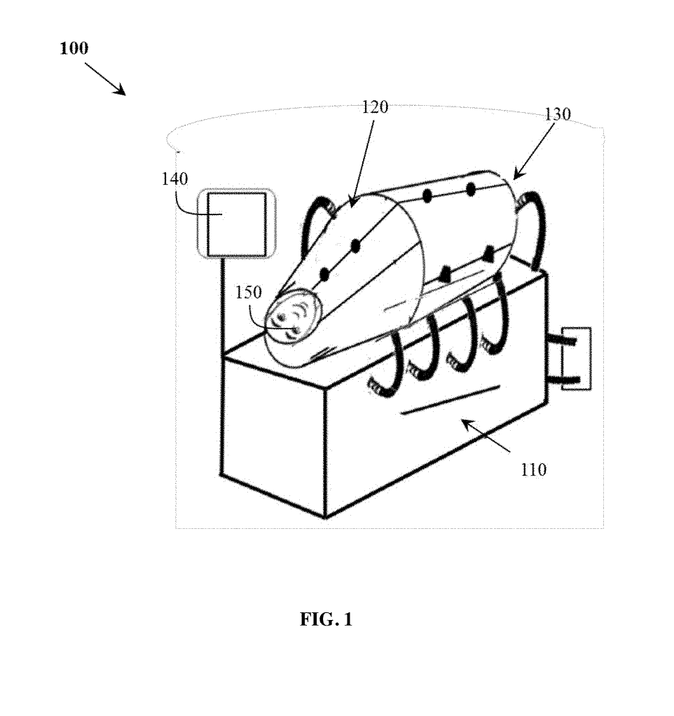

FIG. 1 illustrates a passive anaerobic and aerobic exercise assist system 100, in accordance with an embodiment of the present invention. As shown in FIG. 1, the system 100 comprises an External counter pulsation unit 110. External counter pulsation is a rehabilitation process that provides mechanical external cardiac assistance on a consistent regimen resulting in significant improvement in energy and exercise tolerance much like an exercise program. By restoring oxygenated blood flow, there are abundant benefits to the body of a user 150. This restoration of nutrient rich blood flow revives tissue in parts of the heart and body that have become ‘stunned’ or ‘hibernated’ due to restricted or blocked blood flow. The heart benefits greatly as users will see an improvement in structure, strength (contractility) and a reduction in afterload (workload) due to the reduction in systemic vascular resistance.

Further, the system 100 comprises a perspiration unit 120 connected with the ECP unit 110. The perspiration unit 120 is used as a passive aerobic cardio-vascular work out for the sedentary or physically less active population, using infrared for perspiration. In accordance with an embodiment, the perspiration unit 120 is a Thermal Far Infrared Ray (FIR) system that induces perspiration—vigorous sweating and increased heart rate, similar to those elicited by moderate exercise. Thus, providing the same benefits as those of the moderate exercises to the user 150. Further, the system 100 includes an Electronic Muscle and nerve stimulation unit 130 (EMS/TENS) connected with the perspiration unit 120. Electrical/Electronic muscle stimulation (EMS), also known as neuromuscular electrical stimulation (LAMES) or electro-myo stimulation is the elicitation of muscle contraction using electric impulses. EMS has a potential to serve as a strength training tool for healthy subjects and athletes, a rehabilitation and preventive tool for partially or totally immobilized patients, a testing tool for evaluating the neural and/or muscular function in vivo, and a post-exercise recovery tool for athletes. The EMS technology delivers electrical stimulation directly to the muscles causing them to contraction and relaxation exactly as they do during exercise.

And Transcutaneous electrical nerve stimulation (TENS or TNS) is the use of electric current produced by a device to stimulate the nerves for therapeutic purposes. TENS, by definition, covers the complete range of transcutaneously applied currents used for nerve excitation although the term is often used with a more restrictive intent, namely to describe the kind of pulses produced by portable stimulators used to treat pain. A typical battery-operated TENS unit is able to modulate pulse width, frequency and intensity. Generally, TENS is applied at high frequency (>50 Hz) with an intensity below motor contraction (sensory intensity) or low frequency (<10 Hz) with an intensity that produces motor contraction.

Further the system 100 includes a monitoring device 140 connected to the ECP unit 110, the perspiration unit 120 and the EMS/TENS unit 130. The monitoring device 140 may be selected from one of, but not limited to, a display device such a monitor, a screen or a mobile handheld device such as a mobile phone, a tablet capable of displaying user 150 information in the form of data or images. In accordance with an embodiment, the monitoring device 140 has a touch screen interface capable of receiving external inputs.

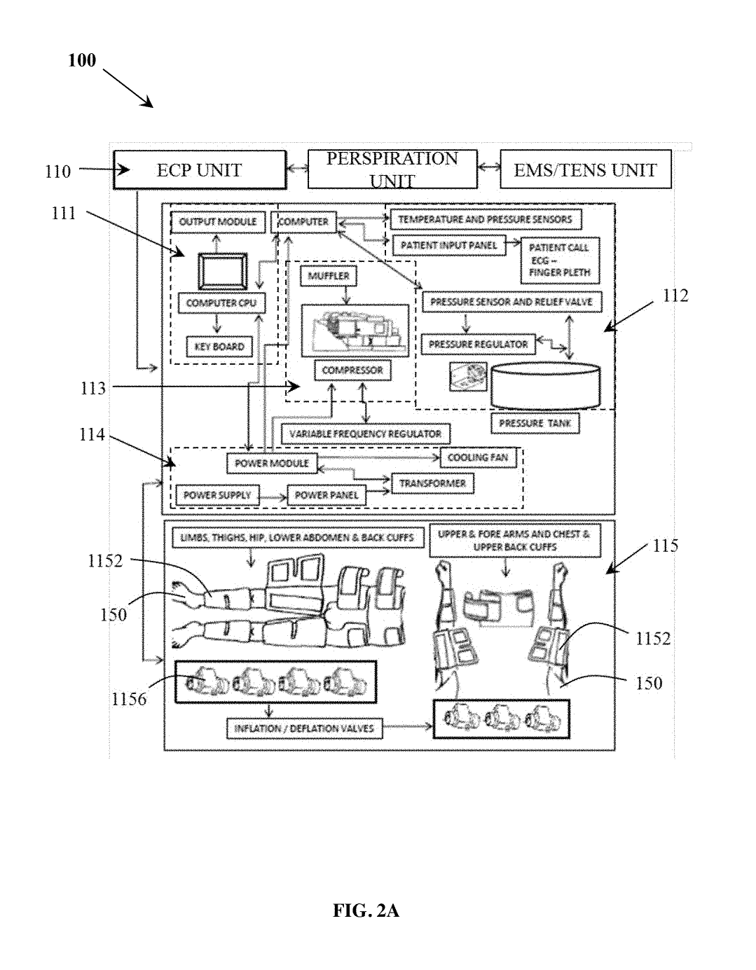

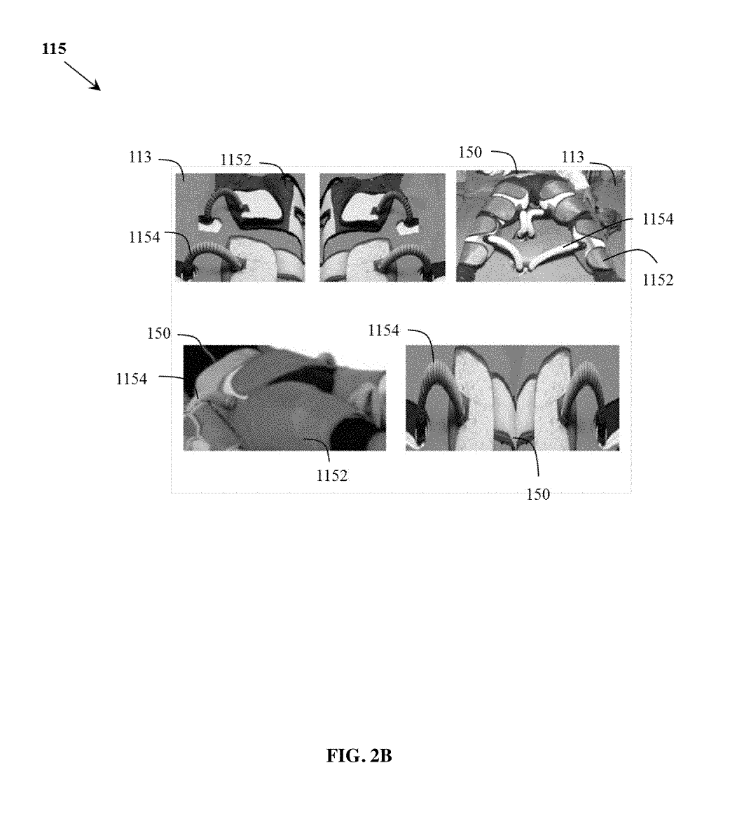

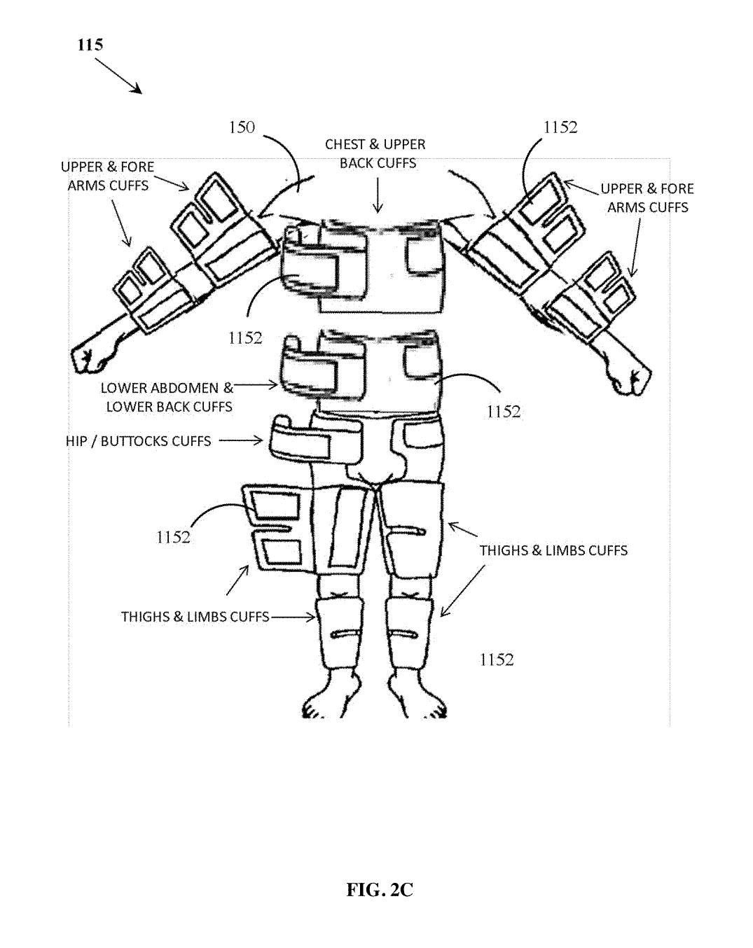

FIG. 2A illustrates the External Counter Pulsation (ECP) unit 110 of the system 100, in accordance with another embodiment of the present invention. As shown in FIG. 2A, the ECP unit 110 includes a first computing device 111. The first computing device 111 further comprises a Central Processing Unit (CPU) operably connected with a display/output means and an input means. The first computing device 111 is selected from one of, but not limited to, a desktop, a laptop, a tablet or mobile computing device. In accordance with an embodiment, the first computing device 111 has a touchscreen interface having an integrated input means.

Also, the ECP unit 110 includes a first control module 112 connected with the first computing device 111. The first control module 112 comprises a plurality of sensors connected to the first computing device 111. The sensors are selected from a group of, but not limited to, a pressure sensor and a temperature sensor. Also, a plurality of valves are connected to a pressure tank and also the first computing device 111. In accordance with an embodiment, the plurality of valves are controlled and operated by first computing device 111. The plurality of valves are selected from a group of, but not limited to, pressure valves and relief valves. The pressure tank is made of, but not limited to a metal, alloy or any rigid material a material capable of withstanding high pressure of air or a gas. Additionally, a pressure regulator connected to the plurality of valves.

Further, the ECP unit 110 comprises a first power module 114 connected with the first control module 112. The air supply module 113 also includes an air compressor. The air compressor is selected from one of, but not limited to, positive displacement compressors or dynamic displacement compressor. Also, a muffler is connected to the compressor for noise reduction. Also, the ECP unit 110 includes a first power module 114 connected with the air supply module 114 and the first computing device 111. Also, the first power module 114 powers the air compressor of the air supply module 114. The first power module comprises a power supply, a power panel connected to the power supply, a transformer connected to the power panel and a cooling fan. Additionally, the ECP unit 110 comprises an implementation section 115 connected with the first power module 114.

FIG. 2B-2C illustrate the implementation section 115 of the ECP unit 110, in accordance with an embodiment of the present invention. As shown in the FIGS. 2B and 2C, the implementation section 115 has a plurality of cuffs 1152. The plurality of cuffs 1152 are capable of being wrapped around a respective body part of the user 150. The body part is one of, but not limited to, the upper arms, fore arms, chest, back, limbs, thigh, calf, hip/buttocks, lower abdomen and lower back. The plurality of cuffs 1152 are made of material having properties like, but not limited to, flexibility and water-resistance. Further, a respective balloon-like element capable of inflating on receiving the air and deflating on releasing the air, is enclosed in each of the plurality of cuffs 1152. Also, a plurality of connection tubes 1154 are provided for connecting the respective plurality of cuffs 1152 to the air supply module 114. An inflation valve and a deflation valve 1156 are also provided on each of the plurality of cuffs 1152.

The first power module 114 is configured to supply electrical power to the air supply module 113 and the first computing device 111. Also, the first computing device 111 is configured to operate the first power module 114. Further, the air supply unit 113 is configured to supply pressurised air to the plurality of cuffs 1152 via the respective plurality of connection tubes 1152 when instructed by the first computing device 111 and extract pressurised air from the plurality of cuffs 1152 via the respective plurality of connection tubes 1152. Then, the first control module 112 is configured to regulate a temperature, an air pressure and a quantity of air supplied from the air supply module 114. Additionally, the plurality of cuffs 1152 are configured to wrap around a body part of the user 150. Also the plurality of cuffs 1152 are configured to inflate on receiving the pressurised air and apply pressure on the body part of the user 150 and also deflate after the pressurised air is extracted from the plurality of cuffs 1152, thereby, releasing the pressure on the body part of the user 150.

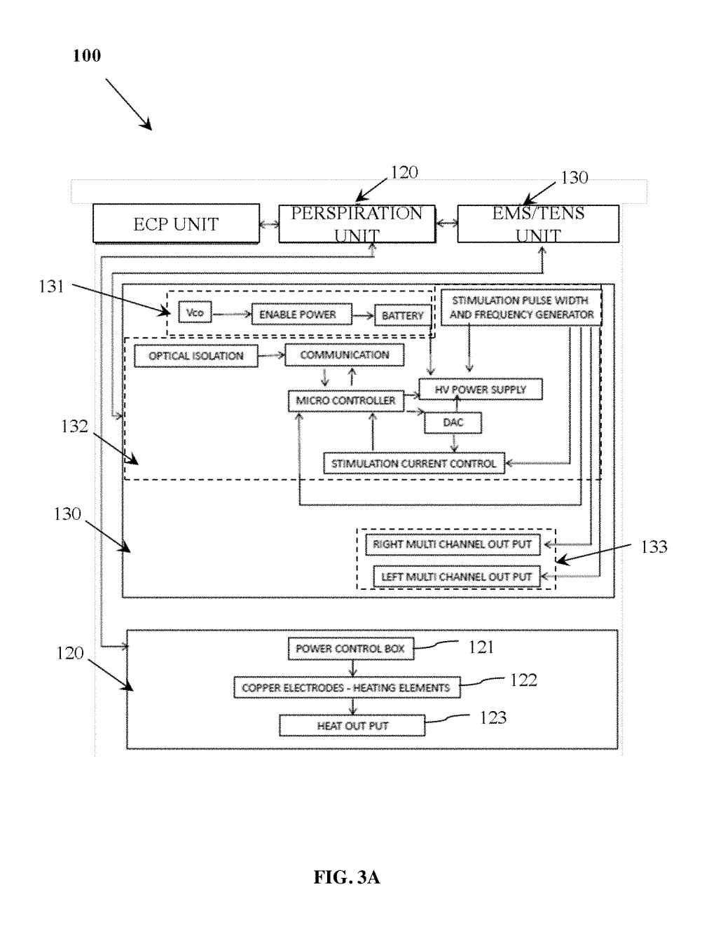

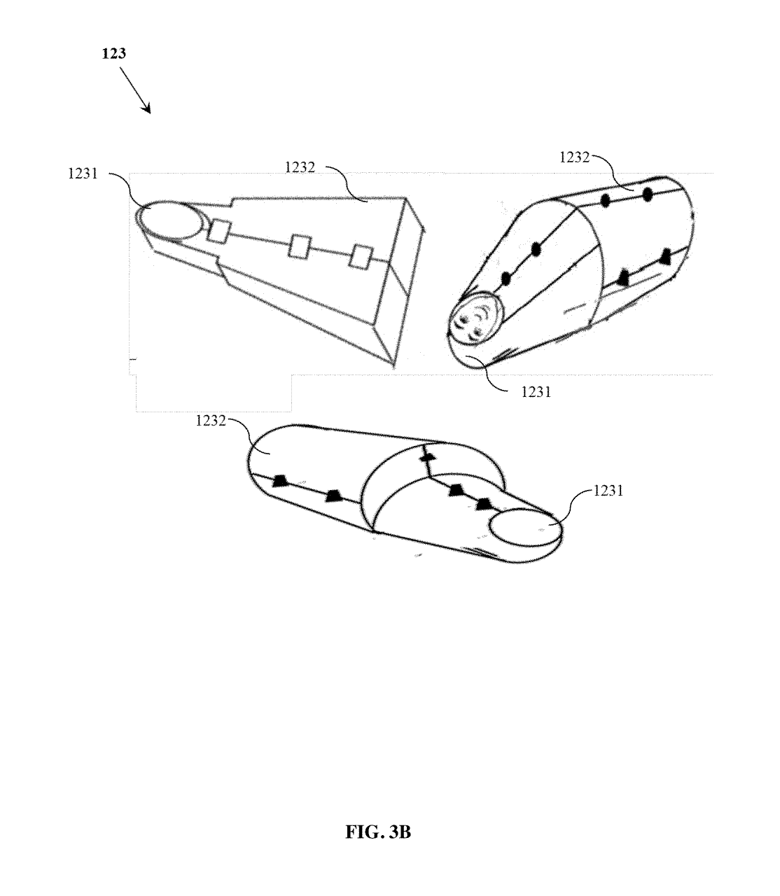

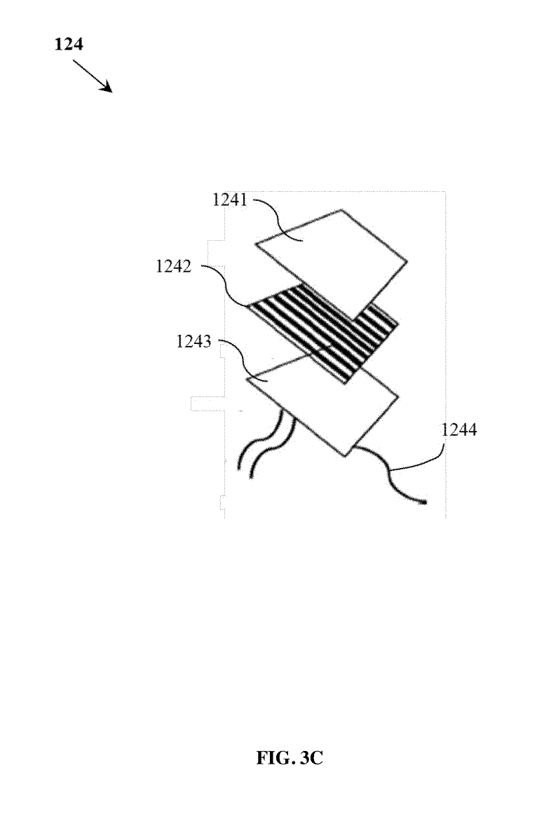

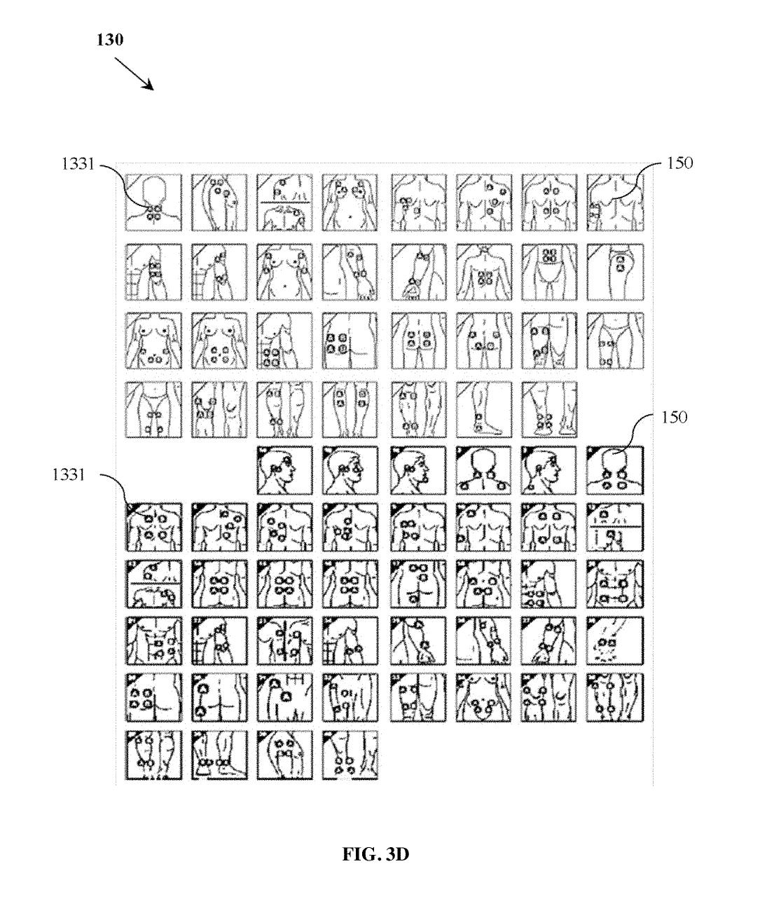

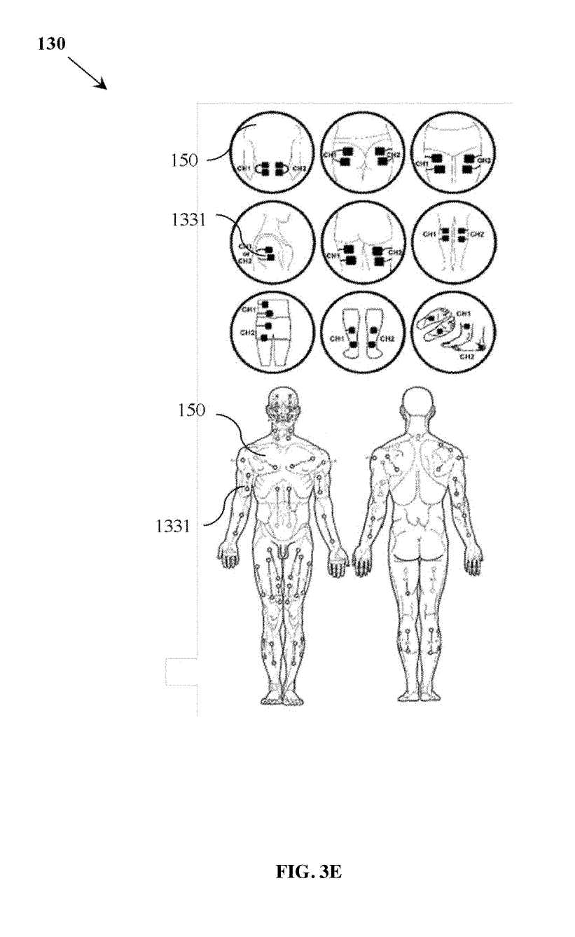

FIG. 3A illustrates the perspiration unit 120 and the Electronic Muscle and Nerve Stimulation (EMS/TENS) unit 130 of the system 100, in accordance with another embodiment of the present invention. The perspiration unit 120 further comprises a heat output zone 123. Referring to FIG. 3B, the heat output zone 123 further has a first housing 1231 and a second housing 1232 connected with the first housing 1231. Unlike traditional perspiration unit, the first housing is provided specifically for receiving the head of the user 150 and the second housing is provided for receiving the body of the user. In accordance with an embodiment, the heat output zone 123 can have a single housing. In one embodiment, the first housing 1231 and the second housing 1232 have a round cross-section. In another embodiment, the first housing 1231 and the second housing 1232 have a rectangular housing and a box-like structure. In yet another embodiment, the first housing 1231 and the second housing 1232 can have different shapes and alignment. Also a thermal blanket 124 is provided in the heat output zone 123. Referring to FIG. 3C, the thermal blanket 124 comprises a first layer 1242 of heating element 122. The heating element 122 comprises a plurality of copper electrodes. Additionally, a second layer 1243 of organic carbon fibre is provided adjacent to the first layer 1242 of heating element 122. And a layer of nylon 1241 covered by a layer of, but not limited to, Polyurethane (PU) fabric or any soft and flexible material, is wrapped around the first layer 1242 and the second layer 1243, thereby forming a blanket. Further, a power control box 121 is connected with the heating element 122.

The power control box 121 is configured to supply electrical power to the heating element 122 and accordingly control the temperature of the heating element 122 and the amount of heat radiated from the heating element 122. Further, the heating element 122 is configured to heat up and provide heat in the heat output zone 123 to the user 150. Also, the thermal blanket 124 is configured to prevent direct contact of the user 150 to the heating element 122.

Returning to FIG. 3A, the Electronic Muscle and Nerve Stimulation (EMS/TENS) unit 130 includes a third power module 131. The third power module 131 further includes a voltage source and/or an electrical power source such as a battery. Further, a third control module 132 is connected with the third power module 131. The third control module 132 comprises a Digital to Analog Convertor (DAC). In electronics, DAC is a device that converts a digital signal into an analog signal. Also, a stimulation current control device is connected with the DAC. Neuromuscular electrical stimulation is well established for rehabilitation and diagnostic purposes. The design of the stimulation control device is in most cases based on voltage controlled (VC) or current controlled (CC) output stages. VC devices are considered to be safer for transcutaneous applications because an electrode error does not lead to dangerous high current density. Whereas, the output force prediction of CC stimulators are more reliable, due to the fact of independency of the electrode impedance. Therefore, the stimulation current control device is preferred.

Additionally, a micro-controller connected with a stimulation current control device is provided in the third control module 132. A microcontroller (or MCU for microcontroller unit) is a small computer on a single integrated circuit. A microcontroller contains one or more CPUs (processor cores) along with memory and programmable input/output peripherals. Further, a communication module connected with the micro-controller. The third control module 132 also includes a stimulation pulse width and frequency generator connected with the stimulation current control device. Further the EMS/TENS unit 130 comprises an output zone 133. In accordance with an embodiment, the output zone 133 comprises a plurality of output channels, for example, left output channel and the right output channel. Referring to FIG. 3D, the output zone 133 further has two or more electrodes 1331. The two or more electrodes 1331 are usually connected to the skin of a user 150. The two or more electrodes 1331 along with the third control module 132 help regulate the process. Further, FIGS. 3D and 3E illustrate electrode placement charts of improved muscle and nerve stimulation (EMS/TENS) unit 130. As shown in FIGS. 3D and 3E, the two or more electrodes 1331 are placed on a plurality of body parts of the user 150.

The third power module 131 is configured to provide electrical current supply to the third control module 132. The third control module 132 is further configured to modulate a pulse width, intensity and frequency of the current supplied and transmit the modulated pulse to the output zone 133 and the output zone 133 via the two or more electrodes 1331, is configured to transmit the modulated pulse to the user 150 for nerve and muscle stimulation.

The method of working of the system 100 is now described. The working of the ECP unit 110 of the system 100 is that the plurality of cuffs 1152 are wrapped around a plurality of body parts of a user 150 such as the upper arms, fore arms, hip/buttocks, thighs, limbs etc. Pressurised air or gas is supplied from the first power module 114 for deflation and inflation in sequence and synchronization for increasing efficiency of the ECP process. The inflated plurality of cuffs 1152 and balloons exert pressure on the respective parts of the body of the user 150, yet with a decreasing pressure grading from distal to proximal parts, so that the blood is driven back to the heart and thence to the trunk and the head. After the completion of inflation, in accordance with predetermined deflation time, and to render deflation of all balloons 1152 that exert pressure on the body. After the completion of deflation, blood is driven to the lower limbs until the next cardiac cycle and the same sequence of events is repeated. After the completion of the inflation, the deflation valves 1156 open simultaneously, thus, gas/air in the balloons and the plurality of cuff 1152 is expelled to the atmosphere. After the completion of deflation, another pulsed signal appears, and the same program is repeated.

Basically, during the process, the respective balloons are pressurized by air, thereby applying pressure to the surface of the enclosed in sequence and synchronization in the following order:

1. limbs and fore arms
2. thighs, hip, lower abdomen and lower back, upper arms
3. the chest and upper back

These features are not available in any other ECP unit. The benefits of ECP are proven in hundreds of clinical trials to be a safe and effective treatment for patients with a variety of medical conditions and also the sedentary and physically less active population. ECP is used to improve the oxygenated blood circulation resulting in a more efficient vascular system to restore nourishment to areas of the body that have slowly deteriorated over time for persons without anaerobic or aerobic exercise activities and will safely result in a significant improvement in overall health.

The working of the perspiration unit 120 is now described. In one embodiment, the perspiration unit 120 is an Infrared Heated Perspiration unit that uses the heating element 122 to emit infrared light experienced as radiant heat which is absorbed by the surface of the skin. Traditional systems heat the body primarily by conduction and convection from the heated air and by radiation of the heated surfaces in an enclosure. The Heated Perspiration unit 120 FIR (Far Infrared) radiant heat warms the body and causes increased blood circulation and lowers blood pressure. Several studies have looked at using infrared systems in the treatment of chronic health problems, such as high blood pressure, congestive heart failure and rheumatoid arthritis, and found some evidence of benefit.

In another embodiment, the perspiration unit 120 is a sauna wrap low-voltage FIR device, with multiple body heating zones simultaneously controlled. The direct heat from the thermal blanket 124 allows for gentle radiant relaxation of the muscles and controlled perspiration. The power control box 121 adjusts and controls the temperature and timer settings of the thermal blanket 124. The Far-infrared system 100 generates heat together with far infrared ray therapy function. For example: When the temperature reaches 75 degrees Celsius (167 degrees Fahrenheit (F)), the energy will infiltrate into 4 cm to 7 cm deep of the muscles and skin. The perspiration unit 120 has an adjustable heat-treatment function between 25 and 75 degrees Celsius (C).

Further, EMS/TENS unit 130 works in a manner that the impulses are generated by the third control module 132 and delivered through the output zone 133 via two or more electrodes 1331 on the skin of a user 150 in direct proximity to the muscles to be stimulated. The impulses mimic the action potential coming from the central nervous system 100, causing the muscles to contract. The two or more electrodes 1331 are generally pads that adhere to the skin. The use of EMS has been cited by sports scientists as a complementary technique for sports training. During conscious exercise and sport, the brain's messages are transmitted to the muscle's motor controls through the nerve-tract by electrical impulses. The EMS technology (Electric Muscle Stimulation) delivers electrical stimulation directly to the muscles causing them to contraction and relaxation exactly as they do during exercise. EMS/TENS unit 130 is suitable for the training of different separated muscle groups through the plurality of output channels in pairs and training through an individual output channel is also possible. Muscle groups can be, but not limited to, lower back, upper back, biceps, triceps, pectoral, abs, gluteus, waist, quadriceps, hamstrings, calf, lower back and upper back. The EMS/TENS unit 130 is designed in such a manner that all the skeletal muscles can be wired simultaneously with plurality of output channels and two or more electrodes 1331 and exercised for maximum benefit in a variety of ways.

The present invention offers a number of advantages. The system 100 is an integrated system 100 housing the External Counter Pulsation (ECP) unit, Electrical/Electronic Muscle and Nerve Stimulation (EMS/TENS) unit and the Perspiration unit 120, enhanced into a single apparatus/system 100. There is no similar predicate device anywhere in the market. Moreover, each of the ECP unit 110, the perspiration unit 120 and the EMS/TENS unit 130 are enhanced and improved from those known in the art to increase industrial applicability. The therapies are executed together (simultaneously). Therefore, the sedentary or the physically less active population can take care of their health without the physical workout. The system 100 is portable and may also be arranged within a moving vehicle, such as a van/bus/etc., (a mobile therapy center for home/work place visits), an airplane, or a ship, a yacht and boats. This system 100 presents a potential and of substantial importance in preventing impairment of human health, due to a sedentary life style.

The terms and descriptions used herein are set forth by way of illustration only and are not meant as limitations. Examples and limitations disclosed herein are intended to be not limiting in any manner, and modifications may be made without departing from the spirit of the present disclosure. Those skilled in the art will recognize that many variations are possible within the spirit and scope of the disclosure, and their equivalents, in which all terms are to be understood in their broadest possible sense unless otherwise indicated.

Various modifications to these embodiments are apparent to those skilled in the art from the description and the accompanying drawings. The principles associated with the various embodiments described herein may be applied to other embodiments. Therefore, the description is not intended to be limited to the embodiments shown along with the accompanying drawings but is to be providing broadest scope of consistent with the principles and the novel and inventive features disclosed or suggested herein. Accordingly, the disclosure is anticipated to hold on to all other such alternatives, modifications, and variations that fall within the scope of the present disclosure and appended claims.

1. A passive anaerobic and aerobic exercise assist system (100), the system (100) comprising:
an External Counter Pulsation (ECP) unit (110), the ECP unit (110) including: a first computing device (111);
a first control module (112) connected with the first computing device (111); an air supply module (113) connected with the first control module (112);
a first power module (114) connected with the air supply module (113) and the first computing device (111); and
an implementation section (115) connected with the air supply module (113), the implementation section (115) having a plurality of cuffs (1152) and a plurality of connection tubes (1154) connecting the respective plurality of cuffs (1152) to the air supply module (113);
a perspiration unit (120) connected with ECP unit (110), the perspiration unit (120) including:
an heat output zone (123), the heat output zone (123) further having a first housing (1231), for receiving a head of the user and a second housing (1232) connected with the first housing (1231), for receiving a body of the user;
a thermal blanket (124) provided in the heat output zone (133);
a heating element (122) disposed in the thermal blanket (124); and
a power control box (123) connected with the heating element (122);
an Electronic Muscle and Nerve Stimulation (EMS/TENS) unit (130) connected with the perspiration unit (120), the EMS/TENS unit (130) including:
a third power module (131);
a third control module (132) connected with the third power module (131);
and
an output zone (133) connected with the third control module (132);
a monitoring device (140) connected with the ECP unit (110), the
perspiration unit (120) and the EMS/TENS unit (130);
wherein the first power module (114) is configured to supply electrical power to the air supply unit (113) and the first computing device (111);
wherein the first computing device (111) is configured to operate the air supply module (120)
wherein the air supply unit (113) is configured to:
supply pressurised air to the plurality of cuffs (1152) via the respective plurality of connection tubes (1154) when instructed by the first computing device (111);
extract pressurised air from the plurality of cuffs (1152) via the respective plurality of connection tubes (1154) when instructed by the first computing device (111);
wherein the first control module (114) is configured to regulate a temperature, an air pressure and a quantity of air supplied from the air supply module (113);
wherein the plurality of cuffs (1152) are
configured to: wrap around a body part
of the user (150);
inflate on receiving the pressurised air and apply pressure on the body part of the user (150);
deflate after the pressurised air is extracted from the plurality of cuffs (1152) and release the pressure on the body part of the user (150);
wherein the power control box (121) is configured to supply electrical power to the heating element (122) and accordingly control the temperature of the heating element
(122) and the amount of heat radiated from the heating element (122);
wherein the heating element (122) is configured to heat up and provide heat in the heat output zone (123) to the user (150);
wherein the thermal blanket (124) is configured to prevent direct contact of the user
(150) to the heating element (122);
wherein the third power module (131) is configured to provide electrical current supply to the third control module (132);
wherein the third control module (132) is configured to modulate a pulse width, intensity and frequency of the current supplied and transmit the modulated pulse to the output zone (133); and
wherein the output zone (133) is configured to transmit the modulated pulse to the user (150) for nerve and muscle stimulation.

2. The system (100) as claimed in claim 1, wherein the first control module (112) comprises a plurality of sensors connected to the first computing device (111), a plurality of valves connected to a pressure tank and the first computing device (111) and a pressure regulator connected to the plurality of valves.

3. The system (100) as claimed in claim 1, wherein the air supply module (113) comprises an air compressor and a muffler connected to the compressor.

4. The system (100) as claimed in claim 1, wherein the first power module (114) comprises a power supply, a power panel connected to the power supply, a transformer connected to the power panel and a cooling fan.

5. The system (100) as claimed in claim 1, wherein the thermal blanket (124) comprises of a first layer (1242) of heating element (122), a second layer (1243) of organic carbon fibre adjacent to the first layer (1242) of heating element and a layer of nylon (1241) covered by a layer of Polyurethane (PU) fabric wrapped around the first layer (1242) and the second layer (1243), thereby forming a blanket.

6. (canceled)

7. The system (100) as claimed in claim 1, wherein the third control module (132) comprises: a Digital to Analog Convertor (DAC);
a stimulation current control device connected with the DAC;
a micro-controller connected with a stimulation current control device; a communication module connected with the micro-controller; and
a stimulation pulse width and frequency generator connected with the stimulation current control device.

8. The system (100) as claimed in claim 1, wherein the output zone (133) comprises a plurality of output channels.

9. The system (100) as claimed in claim 1, wherein the heating element (122) comprises a plurality of copper electrodes.

10. The system (100) as claimed in claim 5, wherein the heating element (122) comprises a plurality of copper electrodes.
